(19) United States
(12) Patent Application Publication
(10) Pub. No.: US 2006/0165740 A1
(43) Pub. Date: Jul. 27, 2006

(54) Perfume delivery system

(75) Inventor: Peter Frank, Powell, OH (US)

Correspondence Address: FITCH, EVEN, TABIN & FLANNERY, P. O. BOX 65973, WASHINGTON, DC, 20035, US

(73) Assignee: Goldschmidt Chemical Corporation, Hopewell, VA (US)

(21) Appl. No.: 11/335,583

(22) Filed: Jan. 20, 2006

Related U.S. Application Data
(60) Provisional application No. 60/645,596, filed on Jan. 24, 2005.

(51) Int. Cl. A61K 8/73

(52) U.S. Cl. 424/401; 424/70.13; 512/1

(57) ABSTRACT

Perfume delivery systems, comprising insoluble carrier particles with surface silanols, which have been grafted with an organosilane and carry amino functional groups, a polymer with positively charged functional groups added to said carrier particles and a fragrance adsorbed to or absorbed into the carrier particles. The perfume delivery systems impart a long lasting fragrance to fabric treated with compositions containing the perfume delivery system.

### CROSS REFERENCE TO RELATED APPLICATIONS

The present application claims priority to and the benefit of U.S. provisional application 60/645,596, filed on Jan. 24, 2005, the contents of which are hereby incorporated by reference in its entirety.

### FIELD OF THE INVENTION

The invention relates to perfume delivery systems, comprising insoluble carrier particles with surface silanols, which have been grafted with an organosilane and which carry amino groups, a polymer carrying positively charged functional groups and a fragrance adsorbed to or absorbed into the carrier particles. The perfume delivery systems impart a long lasting fragrance to fabric treated with compositions containing the perfume delivery system.

### BACKGROUND OF THE INVENTION

Fabric care products such as detergents or fabric softeners usually contain a perfume. The perfume does not only mask unpleasant odors of some of the components of the fabric care product but also impart fabrics treated with the fabric care product with a pleasing fragrance.

Perfumes are among the most expensive ingredients of fabric care products and therefore it is desirable to have as much as possible of the perfume contained in the fabric care product remain in the fabric treated with the fabric care product. This is particularly important for fabric care products which are used for treating fabrics in an aqueous process such as in laundry detergents or rinse cycle fabric softeners. For these products there is a need for perfume delivery systems that are substantive on fibers, which means they will stick to the fibers during the fabric treatment and will not be lost with a wash liquor or rinse liquor. Such a perfume delivery system should also provide a delayed release of perfume to impart the treated fabric with a long lasting fragrance, starting with a moderate level of fragrance intensity that is not perceived as offensive.

GB 1 306 924 discloses finely divided silica and finely divided silica gel as carrier particles for perfume oils. With these carrier particles, liquid perfume oils can be formulated as free flowing powders comprising up to 70 wt.-% of the perfume oil.

U.S. Pat. No. 5,840,668 discloses perfumed laundry detergent powders. The disclosed detergents contain a perfume on carrier system comprising amorphous silica as the carrier. The experiments described in column 7, line 58 to column 9, line 26 demonstrate that perfume adsorbed onto carrier particles of silica is rapidly released into an aqueous wash liquor in the presence of only small amounts of surfactants. Therefore, such a perfume delivery system will not be efficient in delivering a perfume to a fabric treated with the disclosed detergent.

U.S. Pat. No. 4,954,285 discloses the incorporation of such silica particles having a perfume adsorbed thereon into solid dryer-activated fabric softener compositions. The document discloses in column 4, lines 53 to 55 that the perfumed silica particles will release perfume when they are wetted with an aqueous fluid. In accordance with this teaching, the document discloses particles of the fabric softener composition comprising perfumed silica particles having an additional water insoluble coating for the application of the fabric softener in an aqueous process.

U.S. Pat. No. 4,954,285 discloses a perfume containing carrier consisting of particles of a smectite type clay or a zeolite with a perfume absorbed into the particle, having a coating of a fabric adhesive agent, which is preferably a quaternary ammonium compound. The perfume delivery system is used in a laundry detergent and the document discloses an increased level of fragrance for fabrics washed with such a laundry detergent compared to fabrics washed with a laundry detergent containing the perfume without a carrier system. However, the perfume delivery system disclosed in U.S. Pat. No. 4,536,315 still has the drawback that surfactants or dispersants can easily remove the coating of the particles during the process of treating the fabrics, which will diminish the efficiency of perfume delivery.

U.S. Pat. No. 5,476,660 discloses compositions for depositing an active substance, such as a perfume, onto a target surface, such as a fabric, containing carrier particles with a cationic surface, having positively charged organocarbyl groups, and an active substance absorbed or adsorbed by the carrier particles. The carrier particles can be made by coating a solid material like porous silica, zeolite or latex particles with a polymer, which has pendant positively charged groups. An alternative way of making the carrier particles is by grafting a solid material, which has surface reactive groups, with one or more polymers containing difunctional organocarbyl groups.

U.S. Pat. No. 6,020,302 discloses color care compositions comprising a polymer with a polyamine backbone modified via quaternization, substitution or oxidation and optionally a perfume protected by carrier materials such as zeolites, starch, cyclodextrin or wax. The document contains no teaching on the fragrance intensity of fabrics treated with such compositions.

It has now been found that a perfume delivery system, comprising insoluble carrier particles, a polymer carrying positively charged functional groups and a fragrance adsorbed to or absorbed into the carrier particles, can be improved by using carrier particles with surface silanols and grafting the surface silanols with at least one organosilane which carries amino groups. Such perfume delivery systems comprising carrier particles grafted with an organosilane with amino groups surprisingly impart a stronger and longer lasting fragrance to fabrics treated with compositions containing the perfume delivery system compared to perfume delivery systems comprising carrier particles that are not grafted.

### SUMMARY OF THE INVENTION

The present invention relates to a perfume delivery system comprising as a first component water insoluble carrier particles having surface silanol groups, wherein at least part of said silanol groups are substituted with organic residues by grafting with at least one organosilane and wherein at least part of said organic residues carry amino groups, as a second component at least one polymer which carries positively charged functional groups and as a third component a fragrance adsorbed to or absorbed into said carrier particles.

The invention also provides a process for making a perfume delivery system of the present invention comprising the steps reacting water insoluble carrier particles having surface silanol groups with an organosilane having at least one organic residue comprising at least one amino group to obtain carrier particles, wherein at least part of said silanol groups are substituted with organic residues carrying said amino groups, adding at least one polymer carrying positively charged functional groups to said carrier particles, and contacting the obtained particles with a fragrance to adsorb said fragrance onto said particles or absorb said fragrance into said particles.

The invention also provides fabric softening compositions comprising the perfume delivery system of the present invention and one or more fabric softening active quaternary ammonium compounds, as well as laundry detergent compositions, comprising the perfume delivery system of the present invention and one or more surfactants.

### DETAILED DESCRIPTION OF THE INVENTION

A. Carrier Particles

The perfume delivery system of the invention comprises water insoluble carrier particles which initially have surface silanol groups. Such surface silanol groups are hydroxy groups directly bonded to a silicon atom of the carrier particles, which are accessible on the surface and can undergo condensation reactions. The carrier particles may be both inorganic materials or hybrid organic-inorganic polysiloxanes carrying surface silanol groups. Preferably, the carrier particles are inorganic particles selected from silicas, silica gels, silicates or aluminum silicates. The carrier particles of the perfume delivery system of the invention can also be mixtures of these materials. The silicates and aluminum silicates used as carrier particles for the invention preferably contain alkali metal ions or alkali earth metal ions to compensate any extra negative charge of the material. Preferably, negative charges are compensated by sodium ions.

In a preferred embodiment of the invention, the carrier particles are silicas, selected from the group comprising of precipitated silicas, fumed silicas and silica gels. In another preferred embodiment of the invention, the insoluble carrier particles are aluminum silicates with a zeolite structure. Most preferably, the zeolite is a large pore zeolite selected from the group comprising of zeolite X, zeolite Y and dealuminated zeolite Y.

The water insoluble carrier particles preferably have a high specific surface area of more than 30 m²/g and preferably more than 100 m²/g. The carrier particles may be porous particles, such as precipitated silicas, where the specific surface is largely due to the pores of the particles. The water insoluble carrier particles may also be non-porous particles, such as fumed silicas, where the particles are composed of small primary particles having a high geometric outer surface.

The insoluble carrier particles may be small size particles with a particle size in the range of 0.1 to 10 μm. Alternatively, large size particles with a particle size in the range of 10 to 100 μm may be used, which are preferably attained by agglomeration of smaller size particles. Small size carrier particles are preferred, if the perfume delivery system is to be used in a liquid formulation having low viscosity, to avoid settling of the particles. Large size particles are preferred for a convenient handling of the perfume delivery system and to avoid dust formation during the handling.

The surface silanol groups of the insoluble carrier particles are partially or completely substituted with organic residue by a grafting reaction with at least one organosilane. The term organosilane here stands for a silicon compound that carries at least one organic residue bonded to a silicon atom through a silicon-carbon bond and which carries at least one reactive group bonded to silicon that is capable of reacting with a silanol group in a grafting reaction. A grafting reaction is a reaction that forms a covalent Si—O—Si linkage between a silicon atom of the carrier particle and a silicon atom of the organosilane. The grafting reaction leads to a permanent covalent bonding of the organic residue of the organosilane to the surface of the carrier particles.

The organosilanes used for grafting preferably comprise two or three functional groups that are reactive in the grafting reaction, such as chloride, alkoxide or hydroxide bonded to silicon, to enable the formation of multiple linkages between the organosilane and the particle surface in the grafting reaction. Mixtures of two or more organosilanes may be used to obtain the desired composition of organic residue grafted to the carrier particle surface.

At least part of the organic residues grafted to the carrier particle surface carry amino groups. Preferably, at least part of these amino groups are primary amino groups. In a preferred embodiment, each of the organic residues carries at least one amino group. The organic residues may carry one amino group per residue or several amino groups per residue.

The amino group may already be comprised in the organosilane before the organosilane is reacted with the surface silanol groups of the carrier particles in the grafting reaction. In an alternative embodiment, the carrier particles are grafted with an organosilane which carries one or more functional groups other than amino groups which are converted to amino groups after the silane has been grafted onto the carrier particle surface. An example of this embodiment is a carrier particle, which is first grafted with an organosilane comprising one or more epoxy groups and which after the grafting reaction is reacted with a primary or secondary amine to convert at least part of the epoxy groups to the corresponding vicinal hydroxyamine groups.

The size and the composition of the organic residues grafted onto the surface of the carrier particles may be selected in a wide range, as long as at least part of the organic residues carry amino groups. Preferably, the organic residues comprise from 2 to 20 carbon atoms.

It is preferred to select the carrier particle and the at least one organosilane in such a manner as to provide, after grafting, a carrier particle that has a hydrophilic surface and therefore will be wetted when in contact with water. Carrier particles with a hydrophilic surface have the advantage of being easily dispersed in aqueous formulations.

B. Positively Charged Polymer

The perfume delivery system of the invention further comprises at least one polymer carrying positively charged functional groups, which is added to the carrier particles.

The term polymer as used in this invention stands for a molecule made up of one or more repetitive monomer units which comprises at least 10 identical repetitive monomer units. The term polymer encompasses both homopolymers made up of a single monomer unit or copolymers made up of two or more different monomer units. Such copolymers may be random copolymers with a statistical distribution of different monomer units, regular copolymers with alternating monomer units or block copolymers with alternating homopolymer blocks of different monomer units. The term polymer as used in this invention also encompasses any polymer that is modified by functional groups grafted onto the polymer molecule by covalent bonding.

The polymer used in the perfume carrier of the invention carries at least one kind of positively charged functional groups. Suitable positively charged functional groups are ammonium, phosphonium, sulfonium, amidinium, guanidinium or pyridinium functional groups. Preferably, the positively charged functional groups are functional groups with a permanent positive charge independent of the pH value of the medium surrounding the polymer. Most preferably, the positively charged functional groups are quaternary ammonium groups. The positive charges of the functional groups are conveniently compensated by counter ions, such as chloride, bromide, sulfate, phosphate, carbonate, hydrogencarbonate, methylsulfate or the like.

Suitable polymers may be obtained by polymerizing one or more kinds of monomers carrying a positive charge, optionally with one or more comonomers which do not carry a positive charge. A suitable example of a polymer composed of only one kind of monomer is poly-diallyldimethylammonium chloride, known as polyquaternium-6. Examples of suitable copolymers composed of both monomers carrying a positive charge and monomers not carrying a positive charge are polyquaternium-5, polyquaternium-7 and polyquaternium-22.

Alternatively, suitable polymers may be obtained by reacting a polymer, which does not contain a substantial number of positively charged functional groups, with a reagent, which reacts with functional groups of the polymer to generate positively charged functional groups covalently linked to the polymer. Such polymers may be obtained for example by reacting a polymer comprising amino functional groups with an alkylating agent, such as dimethylsulfate, diethylsulfate, dimethylcarbonate, methyl chloride, methyl bromide or benzyl chloride. Alternatively, such polymers may be obtained by reacting a polymer comprising amino or hydroxy functional groups with a quaternary ammonium compound comprising an epoxy or chlorohydrin functional group, such as trimethyl-1-(2,3-epoxypropyl)ammonium chloride or trimethyl-1-(3-chloro-2-hydroxypropyl)ammonium chloride. A further way of preparing such polymer is by reacting a polymer having easily abstractable hydrogen atoms with diallyldimethylammonium chloride in the presence of a radical starter.

Preferably, the polymer carrying positively charged functional groups is composed of one or more carbohydrate monomer units, such as for example glucose, fructose, arabinose, xylose, fucose, galactose, mannose, galacturonic acid, glucuronic acid, mannuronic acid, guluronic acid, galactosamine or glucoseamine. Most preferred are polymers obtained from starch, cellulose, guar gum or locust bean gum or from modified starches or celluloses, such as hydroxymethyl starch, hydroxyethyl starch, carboxymethyl starch, hydroxymethyl cellulose, hydroxyethyl cellulose or carboxymethyl cellulose by reaction with a quaternary ammonium compound comprising an epoxy or chlorohydrin functional group, such as trimethyl-1-(2,3-epoxypropyl)ammonium chloride or trimethyl-1-(3-chloro-2-hydroxypropyl)ammonium chloride or by reaction with diallyldimethylammonium chloride in the presence of a radical starter. Starches from any kind of source, such as potato starch, corn starch, wheat starch, tapioka starch, sago starch or rice starch, can be used. An example of such a most preferred polymer is the reaction product of hydroxyethyl cellulose with trimethyl-1-(2,3-epoxypropyl)ammonium chloride, known as polyquaternium-10, available from National Starch & Chemical under the trade name Celquat SC-240C. Another example is the reaction product of hydroxyethyl cellulose with diallyldimethylammonium chloride, known as polyquatemium-4, available from National Starch & Chemical under the trade name Celquat H-100. The most preferred polymers obtained from modified starches or celluloses have the advantage of being readily biodegradable.

The polymer carrying positively charged functional groups preferably has a molecular weight in the range of from 1,000 to 10,000,000 g/mol and more preferably 5,000 to 5,000,000 g/mol.

The weight ratio of polymer to carrier particles is preferably selected to be from 0.001 to 0.5, more preferably from 0.005 to 0.2 and most preferably from 0.01 to 0.1.

C. Fragrance

The perfume delivery system of the invention further comprises a fragrance adsorbed to or absorbed into the water insoluble carrier particles. The fragrance comprises one or more fragrant compounds and may in addition comprise one or more suitable solvents and further additives, such as antioxidants. The fragrance is applied to the carrier particles in a liquid state, either as such or as a solution in one or more suitable solvents. Application of the fragrance onto the carrier particles may be achieved by any suitable process, such as spraying the fragrance or a fragrance solution onto the carrier particles in a mixer or in a fluidized bed. If the perfume delivery system comprises nonporous carrier particles, the fragrance will be adsorbed to the surface of such carrier particles. If the perfume delivery system comprises porous carrier particles, most of the fragrance will be absorbed into the pores of the carrier particles. The composition of the fragrance and the nature of the fragrant compounds can be selected within a wide range and is not limited, as long as the fragrant compounds are sufficiently stable in contact with the material of the water insoluble carrier particles. If the perfume delivery system is intended to be used in an aqueous formulation or for applications in aqueous systems, the fragrant compounds contained in the fragrance are preferably selected from compounds having a low solubility in water.

Suitable fragrant compounds are for example adoxal (2,6,10-trimethyl-9-undecen-1-al), amyl acetate, amyl salicylate, anisic aldehyde (4-methoxy benzaldehyde), bacdanol (2-ethyl-4-(2,2,3-trimethyl-3-cyclopenten-1-yl)-2-buten-1-ol), benzaldehyde, benzophenone, benzyl acetate, benzyl salicylate, 3-hexen-1-ol, cetalox (dodecahydro-3A,6,6,9A-tetramethylnaphtho[2,1B]-furan), cis-3-hexenyl acetate, cis-3-hexenyl salicylate, citronellol, coumarin, cyclohexyl salicylate, cymal (2-methyl-3-(4-isopropylphenyl) propionaldehyde), decyl aldehyde, ethyl vanillin, ethyl-2-methyl butyrate, ethylene brassylate, eucalyptol, eugenol, exaltolide (cyclopentadecanolide), florhydral (3-(3-isopropylphenyl) butanal), galaxolide (1,3,4,6,7,8-hexahydro-4,6,6,7,8,8-hexamethylcyclopenta-gamma-2-benzopyran e), gamma-decalactone, gamma-dodecalactone, geraniol, geranyl nitrile, helional (alpha-methyl-3,4-(methylenedioxy)hydrocinnamaldehyde), heliotropin, hexyl acetate, hexyl cinnamic aldehyde, hexyl salicylate, hydroxyambran (2-cyclododecyl-propanol), hydroxycitronellal, iso E super (7-acetyl-1,2,3,4,5,6,7,8-octahydro-1,1,6,7,tetramethyl naphthalene), iso-eugenol, iso-jasmone, koavone (acetyl di-isoamylene), lauric aldehyde, lrg 201 (2,4-dihydroxy-3,6-dimethyl benzoic acid methyl ester), lyral (4-(4-hydroxy-4-methyl-pentyl)-3-cylcohexene-1-carboxaldehyde), majantol (2,2-dimethyl-3-(3-methylphenyl)-propanol), mayol (4-(1-methylethyl)cyclohexane methanol), methyl anthranilate, methyl beta-naphthyl ketone, methyl cedrylone (methyl cedrenyl ketone), methyl chavicol (1-methyloxy-4,2-propen-1-yl benzene), methyl dihydrojasmonate, methyl nonyl acetaldehyde, musk indanone (4-acetyl-6-tert-butyl-1,1-dimethylindane), nerol, nonalactone (4-hydroxynonanoic acid lactone), norlimbanol (1-(2,2,6-trimethyl-cyclohexyl)-3-hexanol), P. T. bucinal (2-methyl-3(4-tert-butylphenyl) propionaldehyde), para-hydroxyphenylbutanone, patchouli, phenyl acetaldehyde, phenyl ethyl acetate, phenyl ethyl alcohol, phenyl ethyl phenyl acetate, phenyl hexanol/phenoxanol (3-methyl-5-phenylpentanol), polysantol (3,3-dimethyl-5-(2,2,3-trimethyl-3-cyclopenten-1-yl)-4-penten-2-ol), rosaphen (2-methyl-5-phenyl-pentanol), sandalwood, alpha-terpinene, tonalid/musk plus (7-acetyl-1,1,3,4,4,6-hexamethyltetralin), undecalactone, undecavertol (4-methyl-3-decen-5-ol), undecyl aldehyde, undecenylic aldehyde, vanillin, allyl amyl glycolate, allyl anthranilate, allyl benzoate, allyl butyrate, allyl caprate, allyl caproate, allyl cinnamate, allyl cyclohexane acetate, allyl cyclohexane butyrate, allyl cyclohexane propionate, allyl heptoate, allyl nonanoate, allyl salicylate, amyl cinnamyl acetate, amyl cinnamyl formate, cinnamyl formate, cinnamyl acetate, cyclogalbanate, geranyl acetate, geranyl acetoacetate, geranyl benzoate, geranyl cinnamate, methallyl butyrate, methallyl caproate, neryl acetate, neryl butyrate, amyl cinnamyl formate, alpha-methyl cinnamyl acetate, methyl geranyl tiglate, mertenyl acetate, farnesyl acetate, fenchyl acetate, geranyl anthranilate, geranyl butyrate, geranyl iso-butyrate, geranyl caproate, geranyl caprylate, geranyl ethyl carbonate, geranyl formate, geranyl furoate, geranyl heptoate, geranyl methoxy acetate, geranyl pelargonate, geranyl phenylacetate, geranyl phthalate, geranyl propionate, geranyl iso-propoxyacetate, geranyl valerate, geranyl iso-valerate, trans-2-hexenyl acetate, trans-2-hexenyl butyrate, trans-2-hexenyl caproate, trans-2-hexenyl phenylacetate, trans-2-hexenyl propionate, trans-2-hexenyl tiglate, trans-2-hexenyl valerate, beta-pentenyl acetate, alpha-phenyl allyl acetate, prenyl acetate, trichloromethylphenylcarbinyl acetate, secondary-n-amyl acetate, ortho-tertiary-amyl-cyclohexyl acetate, isoamyl benzyl acetate, sec-n-amyl butyrate, amyl vinyl carbinyl acetate, amyl vinyl carbinyl propionate, cyclohexyl salicylate, dihydro-nor-cyclopentadienyl acetate, dihydro-nor-cyclopentadienyl propionate, isobornyl acetate, isobornyl salicylate, isobornyl valerate, flor acetate, frutene, 2-methylbuten-2-ol-4-acetate, methyl phenyl carbinyl acetate, 2-methyl-3-phenyl propan-2-yl acetate, prenyl acetate, 4-tert-butyl cyclohexyl acetate, verdox (2-tert-butyl cyclohexyl acetate), vertenex, (4-tert-butylcyclohexyl acetate), Violiff (carbonic acid 4-cycloocten-1-yl methyl ester), ethenyl-iso-arnyl carbinylacetate, fenchyl acetate, fenchyl benzoate, fenchyl-n-butyrate, fenchyl isobutyrate, laevo-menthyl acetate, dl-menthyl acetate, menthyl anthranilate, menthyl benzoate, menthyl-iso-butyrate, menthyl formate, laevo-menthyl phenylacetate, menthyl propionate, menthyl salicylate, menthyl-iso-valerate, cyclohexyl acetate, cyclohexyl anthranilate, cyclohexyl benzoate, cyclohexyl butyrate, cyclohexyl-iso-butyrate, cyclohexyl caproate, cyclohexyl cinnamate, cyclohexyl formate, cyclohexyl heptoate, cyclohexyl oxalate, cyclohexyl pelargonate, cyclohexyl phenylacetate, cyclohexyl propionate, cyclohexyl thioglycolate, cyclohexyl valerate, cyclohexyl-iso-valerate, methyl amylacetate, methyl benzyl carbinyl acetate, methyl butyl cyclohexanyl acetate, 5-methyl-3-butyl-tetrahydropyran-4-yl acetate, methyl citrate, methyl-iso-campholate, 2-methyl cyclohexyl acetate, 4-methyl cyclohexyl acetate, 4-methyl cyclohexyl methyl carbinyl acetate, methyl ethyl benzyl carbinyl acetate, 2-methylheptanol-6-acetate, methyl heptenyl acetate, alpha-methyl-n-hexyl carbinyl formate, methyl-2-methylbutyrate, methyl nonyl carbinyl acetate, methyl phenyl carbinyl acetate, methyl phenyl carbinyl anthranilate, methyl phenyl carbinyl benzoate, methyl phenyl carbinyl-n-butyrate, methyl phenyl carbinyl-iso-butyrate, methyl phenyl carbinyl caproate, methyl phenyl carbinyl caprylate, methyl phenyl carbinyl cinnamate, methyl phenyl carbinyl formate, methyl phenyl carbinyl phenylacetate, methyl phenyl carbinyl propionate, methyl phenyl carbinyl salicylate, methyl phenyl carbinyl-iso-valerate, 3-nonyl acetate, 3-nonenyl acetate, nonane diol-2,3-acetate, nonynol acetate, 2-octyl acetate, 3-octyl acetate, n-octyl acetate, secondary-octyl-iso-butyrate, beta-pentenyl acetate, alpha-phenyl allyl acetate, phenylethyl methyl carbinyl-iso-valerate, phenylethyleneglycol diphenylacetate, phenylethyl ethnyl carbinyl acetate, phenylglycol diacetate, seconday-phenylglycol monoacetate, phenylglycol monobenzoate, isopropyl caprate, isopropyl caproate, ispropyl caprylate, isopropyl cinnamate, para-isopropyl cyclohexyl acetate, propylglycol diacetate, propyleneglycol di-isobutyrate, propyleneglycol dipropionate, isopropyl-n-heptoate, isopropyl-n-hept-1-yne carbonate, isopropyl pelargonate, isopropyl propionate, isopropyl undecylenate, isopropyl-n-valerate, isopropyl-n-valerate, isopropyl-iso-valerate, isopropyl sebacinate, isopulegyl acetate, isopulegyl acetoacetate, isopulegyl isobutyrate, isopulegyl formate, thymyl propionate, alpha-2,4-trimethyl cyclohexane methylacetate, trimethyl cyclohexyl acetate, vanillin triacetate, vanillylidene diacetate, vanillyl vanillate, tert-amyl acetate, caryophyllene acetate, cedrenyl acetate, cedryl acetate, dihydromyrcenyl acetate, dihydroterpinyl acetate, dimethyl benzyl carbinyl acetate, dimethyl benzyl carbinyl isobutyrate, dimethyl heptenyl acetate, dimethyl heptenyl formate, dimethyl heptenyl propionate, dimethyl heptenyl-iso-butyrate, dimethyl phenylethyl carbinyl acetate, dimethyl phenylethyl carbinyl-iso-butyrate, dimethyl phenylethyl carbinyl-iso-valerate, dihydro-nor-dicyclopentadienyl acetate, dimethyl benzyl carbinyl butyrate, dimethyl benzyl carbinyl formate, dimethyl benzyl carbinyl propionate, dimethyl phenylethyl carbinyl-n-butyrate, dimethyl phenylethyl carbinyl formate, dimethyl phenylethyl carbinyl propionate, elemyl acetate, ethinyl cyclohexylacetate, eudesmyl acetate, eugenyl cinnamate, eugenyl formate, iso-eugenyl formate, eugenyl phenylacetate, iso-eugenyl phenylacetate, guaiyl acetate, hydroxycitronellyl ethylcarbonate, linallyl acetate, linallyl anthranilate, linallyl benzoate, linallyl butyrate, linallyl iosbutyrate, linallyl carproate, linallyl caprylate, linallyl cinnamate, linallyl citronellate, linallyl formate, linallyl heptoate, linallyl-N-methylanthranilate, linallyl methyltiglate, linallyl pelargonate, linallyl phenylacetate, linallyl propionate, linallyl pyruvate, linallyl salicylate, linallyl-n-valerate, linallyl-iso-valerate, methylcyclopentenolone butyrate, methyl cyclopentenolone propionate, methyl ethyl phenyl carbinyl acetate, methyl heptinyl carbonate, methyl nicotinate, myrcenyl acetate, myrcenyl formate, myrcenyl propionate, cis-ocimenyl acetate, phenyl salicylate, terpinyl acetate, terpinyl anthranilate, terpinyl benzoate, terpinyl-n-butyrate, terpinyl-iso-butyrate, terpinyl cinnamate, terpinyl formate, terpinyl phenylacetate, terpinyl propionate, terpinyl-n-valerate, terpinyl-iso-valerate, tributyl acetylcitrate, amyl vinyl carbinyl acetate, amyl vinyl carbinyl propionate, hexyl vinyl carbinyl acetate, 3-nonenyl acetate, 4-hydroxy-2-hexenyl acetate, linallyl anthranilate, linallyl benzoate, linallyl butyrate, linallyl iso-butyrate, linallyl carproate, linallyl caprylate, linallyl cinnamate, linallyl citronellate, linallyl formate, linallyl heptoate, linallyl-N-methylanthranilate, linallyl methyltiglate, linallyl pelargonate, linallyl phenylacetate, linallyl propionate, linallyl pyruvate, linallyl salicylate, linallyl-n-valerate, linallyl-iso-valerate, myrtenyl acetate, nerolidyl acetate, nerolidyl butyrate, beta-pentenyl acetate, alpha-phenyl allyl acetate, acetylfuran, allethrolone, allyl-ionone, allyl-pulegone, amyl-cyclopentenone, benzylideneacetone, benzylideneacetophenone, alpha-iso-methyl-ionone, 4-(2,6,6-trimetyl-1-cyclohexen-1-yl)-3-buten-2-one, beta-damascone (1-(2,6,6-trimethylcyclohexen-1-yl)-2-buten-1-one), damascenone (1-(2,6,6-trimethyl-1,3-cyclohexadien-1-yl)-2-buten-1-one), delta-damascone (1-(2,6,6-trimethyl-3-cyclo-hexen-1-yl)-2-buten-1-one), alpha-ionone (4-(2,6,6-trimethyl-1-cyclohexenyl-1-yl)-3-buten-2-one), beta-ionone (4-(2,6,6-trimethyl-1-cyclohexen-1-yl)-3-butene-2-one), gamma-methylionone (4-(2,6,6-trimethyl-2-cyclohexyl-1-yl)-3-methyl-3-buten-2-one), pulegone, acetaldehyde benzyl-beta-methoxyethylacetal, acetaldehyde di-iso-amylacetal, acetaldehyde di-pentanediolacetal, acetaldehyde di-n-propylacetal, acetaldehyde ethyl-trans-3-hexenylacetal, acetaldehyde phenylethyleneglycolacetal, acetaldehydephenylethyl n-propylacetal, cinnamic aldehyde dimethylacetal, acetaldehyde benzyl-beta-methoxyethylacetal, acetaldehyde di-iso-amylacetal, acetaldehyde diethylacetal, acetaldehyde di-cis-3-hexenylacetal, acetaldehyde di-pentanediolacetal, acetaldehyde di-n-propylacetal, acetaldehyde ethyl-trans-3-hexenylacetal, acetaldehyde phenylethyleneglycolacetal, acetaldehyde phenylethyl-n-propylacetal, acetylvanillin dimethylacetal, alpha-amylcinnamic aldehyde di-iso-propylacetal, p-tert-amylphenoxyacetaldehyde diethylacetal, anisaldehyde diethylacetal, anisaldehyde dimethylacetal, iso-apiole, benzaldehyde diethylacetal, benzaldehyde di-(ethyleneglycolmonobutylether)acetal, benzaldehyde dimethylacetal, benzaldehyde ethyleneglycolacetal, benzaldehyde glycerylacetal, benzaldehyde propyleneglycolacetal, cinnamic aldehyde diethylacetal, citral diethylacetal, citral dimethylacetal, citral propyleneglycolacetal, alpha-methylcinnamic aldehyde diethylacetal, alpha-cinnamic aldehyde dimethylacetal, phenylacetaldehyde 2,3-butyleneglycolacetal, phenylacetaldehyde citronellyl-methylacetal, phenylacetaldehyde diallylacetal, phenylacetaldehyde diamylacetal, phenylacetaldehyde dibenzylacetal, phenylacetaldehyde dibutylacetal, phenylacetaldehyde diethylacetal, phenylacetaldehyde digeranylacetal, phenylacetaldehyde dimethylacetal, phenylacetaldehyde ethyleneglycolacetal, phenylacetalde glycerylacetal, citronellal cyclomonoglycolacetal, citronellal diethylacetal, citronellal dimethylacetal, citronellal diphenylethylacetal, geranoxyacetaldehyde diethylacetal, acetone diethylketal, acetone dimethylketal, acetophenone diethylketal, methyl-amyl-catechol ketal, methyl-butyl-catechol ketal, anisaldehyde methylanthranilate, aurantiol (hydroxycitronellal methylanthranilate), verdantiol (4-tert-butyl-alpha-methyldihydrocinnamaldehyde methylanthranilate), vertosine (2,4-dimethyl-3-cyclohexene carbaldehyde), hydroxycitronellal ethylanthranilate, hydroxycitronellal linallylanthranilate, methyl-N-(4-(4-hydroxy-4-methylpentyl)-3-cyclohexenyl-methylidene)anthranilate, methylnaphthylketone methylanthranilate, methyl-nonyl-acetaldehyde methylanthranilate, methyl-N-(3,5,5-trimethylhexylidene) anthranilate, vanillin methylanthranilate, amyl acetate, amyl propionate, anethol, anisic aldehyde, anisole, benzaldehyde, benzyl acetate, benzyl acetone, benzyl alcohol, benzyl butyrate, benzyl formate, benzyl iso-valerate, benzyl propionate, camphor gum, carvacrol, laevo-carveol, d-carvone, laevo-carvone, citral (neral), citronellol, citronellyl acetate, citronellyl isobutyrate, citronellyl nitrile, citronellyl propionate, para-cresol, para-cresyl methyl ether, cyclohexyl ethyl acetate, cuminic alcohol, cuminic aldehyde, cyclal C (3,5-dimethyl-3-cyclohexene-1-carboxaldehyde), para-cymene, decyl aldehyde, dimethyl benzyl carbinol, dimethyloctanol, diphenyl oxide, dodecalactone, ethyl acetate, ethyl acetoacetate, ethyl amyl ketone, ethyl benzoate, ethyl butyrate, ethyl hexyl ketone, ethyl phenyl acetate, eucalyptol, eugenol, fenchyl alcohol, geraniol, geranyl nitrile, hexenol, beta-gamma-hexenol, hexenyl acetate, cis-3-hexenyl acetate, hexenyl isobutyrate, cis-3-hexenyl tiglate, hexyl acetate, hexyl formate, hexyl neopentanoate, hexyl tiglate, hydratropic alcohol, hydroxycitronellal, indole, alpha-irone, isoamyl alcohol, isobutyl benzoate, isomenthone, isononyl acetate, isononyl alcohol, isobutyl quinoline, isomenthol, para-isopropyl phenylacetaldehyde, isopulegol, isopulegyl acetate, isoquinoline, cis-jasmone, lauric aldehyde (dodecanal), ligustral (2,4-dimethyl-3-cyclohexene-1-carboxaldehyde), linalool, linalool oxide, menthone, methyl acetophenone, para-methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, methyl nonyl acetaldehyde, methyl octyl acetaldehyde, methyl salicylate, myrcene, neral, nerol, gamma-nonalactone, nonyl acetate, nonyl aldehyde, allo-ocimene, octalactone, 2-octanol, octyl aldehyde, d-limonene, phenoxy ethanol, phenyl acetaldehyde, phenyl ethyl acetate, phenyl ethyl alcohol, phenyl ethyl dimethyl carbinol, propyl butyrate, rose oxide, 4-terpinenol, alpha-terpineol, terpinolene, tonalid (6-acetyl-1,1,3,4,4,6-hexamethyltetrahydronaphthalene), undecenal, veratrol (1,2-dimethoxybenzene), ambrox (1,5,5,9-tetramethyl-1,3-oxatricyclotridecane), anethole, bacdanol (2-ethyl-4-(2,2,3-trimethyl-3-cyclopenten-1-yl)-2-buten-1-ol), benzyl acetone, benzyl salicylate, butyl anthranilate, calone, cetalox (2-ethyl-4-(2,2,3-trimethyl-3-cyclopenten-1-yl)-2-buten-1-ol), cinnamic alcohol, coumarin, Cyclal C (3,5-dimethyl-3-cyclohexene-1-carboxaldehyde), cymal (2-methyl-3-(4-isopropylphenyl)propionaldehyde), 4-decenal, dihydroisojasmonate, gamma-dodecalactone, ebanol, ethyl anthranilate, ethyl-2-methyl butyrate, ethyl vanillin, eugenol, florhydral (3-(3-isopropylphenyl)butanol), fructone (ethyl-2-methyl-1,3-dioxolane-2-acetate), heliotropin, herbavert (3,3,5-trimethylcyclohexyl ethyl ether), cis-3-hexenyl salicylate, indole, isocyclocitral, isoeugenol, alpha-isomethylionone, keone, lilial (para-tert-butyl alpha-methyl hydrocinnamic aldehyde), linalool, lyral (4-(4-hydroxy-4-methyl-pentyl)-cylcohexene-1-carboxaldehyde), methyl heptine carbonate, methyl anthranilate, methyl dihydrojasmonate, methyl-isobutenyl-tetrahydropyran, methyl beta-naphthyl ketone, methyl nonyl ketone, beta-naphthol methyl ether, nerol, para-anisic aldehyde, para-hydroxyphenyl-butanone, phenylacetaldehyde, gamma-undecalactone and undecylenic aldehyde. Suitable are also fragrant naturally occurring plant and animal oils and exudates and extracts obtained from plant and animal material.

The weight ratio of fragrance to carrier particles may be varied in a wide range and is preferably selected to be from 0.01 to 5 and most preferably 0.2 to 3. The weight ratio is selected, depending on the surface area and on the pore volume of the carrier particles, in such a manner that essentially all of the fragrance is adsorbed to or absorbed into the carrier particles to obtain a perfume delivery system which is a dry, free flowing powder.

D. Process for Making a Perfume Delivery System

The invention also provides a process for making a perfume delivery system of the present invention. This process comprises the steps

a) reacting water insoluble carrier particles having surface silanol groups with an organosilane having at least one organic residue comprising at least one amino group to obtain carrier particles, wherein at least part of said silanol groups are substituted with organic residues carrying said amino groups,
b) adding at least one polymer carrying positively charged functional groups to said carrier particles, and
c) contacting the particles obtained in step b) with a fragrance to adsorb said fragrance onto said particles or absorb said fragrance into said particles.
   The organosilane reacted with the carrier particles in step a) preferably has the formula
   (R¹O)₃₋ₙR²ₙSi(CH₂)₃Z,
   wherein
   R¹ and R² are independently methyl, ethyl, n-propyl or n-butyl,
   n is 0 or 1,
   Z is NR³R⁴,
   R³ and R⁴ are independently hydrogen, methyl, ethyl, C₃₋₂₀ alkyl, C₇₋₂₆ aralkyl,
   (CH₂CH₂O)ₙR⁵ or (CH₂CH₂NH)ₙR⁵,
   m is from 1 to 4, and
   R⁵ is hydrogen, methyl, ethyl, C₃₋₂₀ alkyl or C₇₋₂₆ aralkyl.

The most preferred organosilanes are (MeO)₃Si(CH₂)₃NH₂, (EtO)₃Si(CH₂)₃NH₂, (MeO)₂MeSi(CH₂)₃NH₂, (EtO)₂MeSi(CH₂)₃NH₂, (MeO)₃Si(CH₂)₃NH(CH₂)₃CH₃, (EtO)₃Si(CH₂)₃NH(CH₂)₃CH₃, (MeO)₂MeSi(CH₂)₃NH(CH₂)₃CH₃, (EtO)₂MeSi(CH₂)₃NH(CH₂)₃CH₃, (MeO)₃Si(CH₂)₃NH(CH₂)₂NH₂, (EtO)₃Si(CH₂)₃NH(CH₂)₂NH₂, (MeO)₂MeSi(CH₂)₃NH(CH₂)₂NH₂, (EtO)₂MeSi(CH₂)₃NH(CH₂)₂NH₂, (MeO)₃Si(CH₂)₃NH(CH₂)₂NHCH₂Ph, (EtO)₃Si(CH₂)₃NH(CH₂)₂NHCH₂Ph, (MeO)₂MeSi(CH₂)₃NH(CH₂)₂NHCH₂Ph, (EtO)₂MeSi(CH₂)₃NH(CH₂)₂NHCH₂Ph, (MeO)₃Si(CH₂)₃NH(CH₂)₂NH(CH₂)₂NH₂, (EtO)₃Si(CH₂)₃NH(CH₂)₂NH(CH₂)₂NH₂, (MeO)₂MeSi(CH₂)₃NH(CH₂)₂NH(CH₂)₂NH₂, (EtO)₂MeSi(CH₂)₃NH(CH₂)₂NH(CH₂)₂NH₂, wherein Me is methyl, Et is ethyl and Ph is phenyl.

In step a) of the process, the organosilane is preferably reacted with the carrier particles in a weight ratio of from 1:1 to 1:100. More preferably, the weight ratio of organosilane to carrier particles is from 1:5 to 1:50.

The organosilane can be reacted with the carrier particles in a suitable solvent in the absence of water. The reaction is carried out for a suitable time to achieve grafting of the silane onto the carrier particle. The reaction temperature is selected according to the nature of the reactive groups on the silicon atom of the silane and is preferably in the range of 20 to 100° C.

The organosilane can also be reacted with the carrier particles in the presence of water. In this case, the organosilane or a solution of the organosilane is sprayed onto the carrier particles and the resulting mixture is dried to complete the grafting reaction, preferably at a temperature of from 100 to 200° C., in particular from 100 to 150° C.

In step b), at least one polymer carrying positively charged functional groups is added to the carrier particles obtained in step a). Preferably, the addition of the polymer is performed in a manner to obtain an intimate mixture of the carrier particles and the polymer. If the polymer is a solid, the polymer can be dry mixed with the carrier particles.

In an alternative embodiment, a solution of the polymer in a solvent or a mixture of solvents is added to the carrier particles. In this case water is preferably used as a solvent. The one or more solvents are preferably removed from the resulting suspension by evaporation. Alternatively, the one or more solvents may be also be separated from the carrier particles by suitable mechanical means, such as filtration or centrifugation, after a sufficient amount of the polymer has adsorbed to the carrier particles.

In step c), the contacting of the particles obtained in step b) with a fragrance is preferably carried out by spraying the fragrance or a solution of the fragrance in one or more suitable solvents onto the carrier particles while maintaining the carrier particles in a free flowing state. The spraying can be performed in a mixer, where the particles are moved by mechanical means, or in a fluidized bed, where the particles are moved by a fluidizing gas. If a solvent is used, the solvent may be conveniently removed during the spraying step, although this is not necessary.

E. Fabric Softening Composition

The invention further provides fabric softening compositions comprising the perfume delivery system of the present invention and one or more fabric softening active quaternary ammonium compounds.

A fabric softening active quaternary ammonium compound is a quaternary ammonium compound which when contacted with a fabric will impart a soft touch to the fabric.

Suitable fabric softening active quaternary ammonium compounds are compounds of formula (I)
R⁶₄₋ₘN⁺[(CH₂)ₙ-Q-R⁷]ₘX⁻ (I)
wherein each R⁶ is independently C₁-C₆ alkyl, C₁-C₆ hydroxyalkyl or benzyl and preferably is methyl;
R⁷ is independently hydrogen, C₁₁-C₂₂ linear alkyl, C₁₁-C₂₂ branched alkyl, C₁₁-C₂₂ linear alkenyl or C₁₁-C₂₂ branched alkenyl, with the proviso that at least one of R⁷ is not hydrogen;
Q is independently selected from the units having the formula —O—C(O)—, —C(O)O—, —NR⁸—C(O)—, —C(O)—NR⁸—, —O—C(O)—O—, —CHR⁹—O—C(O)— or —CH(OCOR⁷)—CH₂—O—C(O)—, wherein R⁸ is hydrogen, methyl, ethyl, propyl or butyl and R⁹ is hydrogen or methyl and preferably Q is —O—C(O)— or —NH—C(O)—;
m is from 1 to 4 and preferably 2 or 3;
n is from 1 to 4 and preferably 2; and
X⁻ is a softener compatible anion, for example, chloride, bromide, methylsulfate, ethylsulfate, sulfate or nitrate, preferably chloride or methylsulfate.

The fabric softening active quaternary ammonium compounds of formula (I) can be mixtures of compounds with the number of groups R⁷ per molecule which are not hydrogen ranging from 1 to m. Preferably, such mixtures comprise on average from 1.2 to 2.5 groups R⁷ per molecule which are not hydrogen. More preferably, the amount of non-hydrogen R⁷ groups is from 1.4 to 2.0 and most preferably from 1.6 to 1.9.

The most preferred compounds of formula (I) are the compounds of formulae (II) to (IV):
R⁶N⁺[CH₂CHR⁹OH][CH₂CHR⁹OC(O)R⁷]₂X⁻ (II)
R⁶₂N⁺[CH₂CHR⁹OC(O)R⁷]₂X⁻ (III)
R⁶N⁺[CH₂CHR⁹OH][CH₂CH₂NHC(O)R⁷]₂X⁻ (IV)
wherein R⁶, R⁷ and X have the same meaning as defined for formula (I) above, with the proviso that R⁷ is not hydrogen.

Preferably, the unit —C(O)R⁷ is a fatty acyl moiety. Suitable fatty acyl moieties are derived from natural sources of triglycerides, preferably tallow, vegetable oils, partially hydrogenated tallow and partially hydrogenated vegetable oils. Suitable sources of triglycerides are soy, tallow, partially hydrogenated tallow, palm, palm kernel, rape seed, lard, coconut, canola, safflower, corn, rice and tall oil. The formulator, depending upon the desired physical and performance properties of the final fabric softener, can choose any of the above mentioned sources of fatty acyl moieties, or alternatively, the formulator can mix sources of triglyceride to form a blend.

Those skilled in the art of fats and oils recognize that the fatty acyl composition may vary, as in the case of vegetable oil, from crop to crop, or from variety of vegetable oil source to variety of vegetable oil source. The R⁷ groups are typically mixtures of linear and branched chains of both saturated and unsaturated aliphatic fatty acids.

The fraction of unsaturated groups R⁷ in such mixture is preferably at least 10%, most preferably at least 25% and most preferably from 40% to 70%. The fraction of polyunsaturated groups R⁷ in such mixture is preferably less than 10%, more preferably less than 5% and most preferably less than 3%. Partial hydrogenation can be employed, if required, to minimize the polyunsaturate levels in order to improve the stability (e.g., odor, color, etc.) of the final product. The level of unsaturation, expressed by the iodine value, should preferably be in the range of from 5 to 150 and more preferably in the range from 5 to 50. The ratio of cis and trans isomers of double bonds in the unsaturated groups R⁷ is preferably larger than 1:1 and most preferably in the range 4:1 to 50:1.

Preferred examples of compounds of formula (I) are:
N,N-di(tallowyl-oxy-ethyl)-N,N-dimethyl ammonium chloride;
N,N-di(canolyl-oxy-ethyl)-N,N-dimethyl ammonium chloride;
N,N-di(tallowyl-oxy-ethyl)-N-methyl, N-(2-hydroxyethyl) ammonium methyl sulfate; N,N-di(canolyl-oxy-ethyl)-N-methyl, N-(2-hydroxyethyl) ammonium methyl sulfate;
N,N-di(tallowylamidoethyl)-N-methyl, N-(2-hydroxyethyl) ammonium methyl sulfate;
N,N-di(2-tallowyloxy-2-oxo-ethyl)-N,N-dimethyl ammonium chloride;
N,N-di(2-canolyloxy-2-oxo-ethyl)-N,N-dimethyl ammonium chloride;
N,N-di(2-tallowyloxyethylcarbonyloxyethyl)-N,N-dimethyl ammonium chloride;
N,N-di(2-canolyloxyethylcarbonyloxyethyl)-N,N-dimethyl ammonium chloride;
N-(2-tallowoyloxy-2-ethyl)-N-(2-tallowyloxy-2-oxo-ethyl)-N,N-dimethyl ammonium chloride;
N-(2-canolyloxy-2-ethyl)-N-(2-canolyloxy-2-oxo-ethyl)-N,N-dimethyl ammonium chloride;
N,N,N-tri(tallowyl-oxy-ethyl)-N-methyl ammonium chloride;
N,N,N-tri(canolyl-oxy-ethyl)-N-methyl ammonium chloride;
1,2-ditallowyloxy-3-N,N,N-trimethylammoniopropane chloride; and
1,2-dicanolyloxy-3-N,N,N-trimethylanumoniopropane chloride.

Also suitable as fabric softening active quaternary ammonium compounds are compounds of formula (V)
R⁶²R₂N⁺X⁻ (V)
wherein R⁶, R⁷ and X have the same meaning as defined for formula (I) above, with the proviso that R⁷ is not hydrogen.

Preferred examples of compounds of formula (V) are ditallowdimethylammonium chloride, ditallowdimethylammonium methylsulfate, di(hydrogenatedtallow)dimethylammonium chloride, distearyldimethylammonium chloride and dibehenyldimethylammonium chloride.

Further suitable as fabric softening active quaternary ammonium compounds are compounds of formulae (VI) and (VII):
[R⁷—C(O)NHCH₂CH₂]₂N⁺R⁶[CH₂CH₂OH]X⁻ (VI) 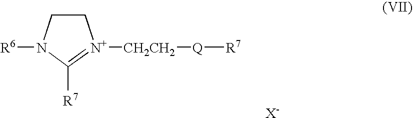
wherein R⁶, R⁷ and X have the same meaning as defined for formula (I) above, with the proviso that R⁷ is not hydrogen, and Q is —O—C(O)— or —NH—C(O)—.

For application as rinse cycle softeners, the fabric softening compositions of the invention preferably comprise from 0.1 to 5 wt.-% of the perfume delivery system, from 1 to 50 wt.-% of fabric softening active quaternary ammonium compounds and water. More preferably, the fabric softening compositions comprise from 0.2 to 2 wt.-% of the perfume delivery system and most preferably from 0.3 to 1.0 wt.-%.

In addition to the perfume delivery system, one or more fabric softening active quaternary ammonium compounds and water, such fabric softening compositions for use as rinse cycle softeners may comprise further additives known from the prior art for formulating aqueous fabric softening compositions, such as viscosity and dispersibility aids, stabilizers, soil release agents, bactericides, nonionic softeners, colorants, preservatives, optical brighteners, opacifiers, fabric conditioning agents, surfactants, anti-shrinkage agents, anti-wrinkle agents, fabric crisping agents, anti-spotting agents, fungicides, anti-corrosion agents and/or antifoam agents. Suitable additives are disclosed in U.S. Pat. No. 6,737,392 in column 8, line 1 to column 14, line 6, and are hereby incorporated by reference.

For application as dryer-added softeners, the fabric softening compositions of the invention preferably comprise a mixture containing from 0.1 to 5 wt.-% of the perfume delivery system and from 1 to 99 wt.-% of fabric softening active quaternary ammonium compounds disposed on an absorbent article. More preferably, the fabric softening compositions comprise from 0.2 to 2 wt.-% of the perfume delivery system and most preferably from 0.3 to 1.0 wt.-%.

Absorbent articles with a fabric softening active material disposed thereon, which are useful as dryer-added softeners and methods for disposing a fabric softening composition on a suitable absorbent article are well known from the prior art. Preferably, the absorbent article has the shape of a sheet comprising a woven or non-woven fiber material. More preferably, the sheet is a paper sheet or a non-woven fleece or a woven cloth made from cellulose, regenerated cellulose or polyester fibers. Suitable sheets of woven and non-woven fiber material and methods for deposing a fabric softening composition on said sheets are disclosed in U.S. Pat. No. 3,686,025, which is incorporated herein by reference. In such an embodiment, the fabric softening composition may be disposed either on the surface of the sheet or preferably in between the fibers of the sheet. In an alternative embodiment, the absorbent article comprises a sponge like or open pore rigid foam material with the fabric softening composition disposed in the pores of the sponge or foam.

In addition to fabric softening active quaternary ammonium compounds, the fabric softening compositions for use as dryer-added softeners may further comprise one or more co-softeners, which are carboxylic acid salts of tertiary amines having the structure
R₁₀R¹¹R¹²NH⁺R¹³COO⁻

wherein R¹⁰ is a long chain alkyl or alkenyl group containing from about 8 to about 30 carbon atoms; R¹¹ and R¹² are the same or different and are selected from the group consisting of alkyl groups containing from 1 to 30 carbon atoms, hydroxyalkyl groups containing from 2 to 30 carbon atoms, and alkyl ether groups of the formula R¹⁴(OCHR¹⁵CH₂)ₙ, wherein R¹⁴ is hydrogen, an alkyl group containing from 1 to 30 carbon atoms or an alkenyl group containing from 3 to 30 carbon atoms, R¹⁵ is hydrogen or methyl and n is from 1 to 30; wherein R¹⁰, R¹¹, R¹² and R¹⁴ chains can be ester interrupted groups; and wherein R¹³ is an alkyl, alkenyl, aryl, alkaryl or aralkyl group comprising 8 to 30 carbon atoms. The amine and the acid, used to form the amine salt may both be of mixed chain lengths rather than single chain lengths and may comprise materials derived from natural fats and oils or synthetic processes which produce a mixture of chain lengths. The co-softeners preferably have a softening point in the range from 35° C. to 100° C. Preferred tertiary amines used as starting materials to form the co-softener tertiary amine salts are lauryldimethylamine, myristyldimethylamine, stearyldimethylamine, tallowdimethylamine, coconutdimethylamine, dilaurylmethylamine, distearylmethylamine, ditallowmethylamine, oleyldimethylamine, dioleylmethylamine, lauryl-di(3-hydroxypropyl)amine, stearyl-di(2-hydroxyethyl)amine, trilaurylamine and laurylethylmethylamine. Preferred carboxylic acids used as starting materials to form the co-softener tertiary amine salts are stearic acid, oleic acid, lauric acid, myristic acid and palmitic acid.

In addition to the perfume delivery system, one or more fabric softening active quaternary ammonium compounds and an absorbent substrate, such fabric softening compositions for use as dryer-added softeners may comprise further additives known from the prior art for formulating fabric softeners, such as nonionic surfactants, fatty acids and alkoxylated fatty acids, stabilizers, soil release agents, bactericides, nonionic softeners, colorants, preservatives, optical brighteners, fabric conditioning agents, surfactants, anti-shrinkage agents, anti-wrinkle agents, fabric crisping agents, anti-spotting agents, fungicides and/or anti-corrosion agents. Suitable additives are disclosed in U.S. Pat. No. 6,737,392 in column 9, line 47 to column 14, line 6, and are hereby incorporated by reference.

The fabric softening compositions of the invention impart a stronger and longer lasting fragrance to fabrics treated with said compositions compared to fabrics treated with compositions comprising perfume delivery systems of the prior art. Such fabric softening compositions may therefore be formulated with a lower amount of fragrant compounds as used in the prior art.

F. Laundry Detergent Compositions

The invention further provides laundry detergent compositions comprising the perfume delivery system of the present invention and one or more surfactants. The term laundry detergent composition as used in this invention encompasses all compositions that can be used to clean fabrics in an aqueous wash liquid.

The laundry detergent compositions of the invention may be solid compositions. Such solid compositions may have the appearance of powders, granulates or molded bodies. Compositions in the form of granulates or molded bodies may comprise the perfume delivery system in the form of particles separate from the granulates or molded bodies. Alternatively, the perfume delivery system may be incorporated into granulates or molded bodies comprising further constituents of the laundry detergent composition. The molded bodies may have the shape of extrudates, pellets, briquettes or tablets. Such molded bodies may be prepared by processes of press agglomeration, such as for example extrusion, briquetting or tabletting. Laundry detergent composition in the form of press molded bodies may contain additional binders to improve the hardness of the molded bodies. However, laundry detergent composition in the form of press molded bodies are preferably made without the use of additional binders with one of the wash active components, preferably a nonionic surfactant, acting as the binder.

In a further embodiment, the laundry detergent compositions of the invention may be liquid or gel compositions with the perfume delivery system of the present invention dispersed in the liquid or gel phase. Apart from the perfume delivery system, further solid components of the detergent may be dispersed in the liquid or gel phase. The rheologic properties of the liquid or gel composition are preferably selected to maintain all solid components dispersed in the liquid or gel phase during storage with no settling of solids. Preferably, the liquid or gel composition shows thixotropic or pseudoplastic flow. Such flow properties may be achieved by additives, such as dispersable clays, in particular montmorillonites; precipitated or pyrogenic silicas; vegetable gums, in particular xanthanes; and synthetic polymeric thickeners, such as vinyl polymers comprising carboxyl groups.

The laundry detergent compositions of the present invention comprise one or more surfactants, preferably anionic, nonionic or cationic surfactants or combinations thereof.

Suitable anionic surfactants are for example surfactants with sulfonate groups, preferably alkylbenzenesulfonates, alkanesulfonates, alpha-olefinsulfonates, alpha-sulfofatty acid esters or sulfosuccinates. Preferred alkylbenzenesulfonates comprise a linear or branched chain alkyl group with 8 to 20 carbon atoms, in particular 10 to 16 carbon atoms. Preferred alkanesulfonates comprise a linear chain alkyl group with 12 to 18 carbon atoms. Preferred alpha-olefinsulfonates are the products of sulfonating alpha-olefins having 12 to 18 carbon atoms. Preferred alpha-sulfofatty acid esters are the products of sulfonating fatty acid esters of fatty acids having 12 to 18 carbon atoms and short chain alcohols selected from methanol, ethanol, 1-propanol and 2-propanol.

A further class of suitable anionic surfactants are surfactants comprising sulfate groups, preferably alkylsulfates and ethersulfates. Preferred alkylsulfates comprise linear chain alkyl group with 12 to 18 carbon atoms. Suitable are also beta-branched alkylsulfates and alkylsulfates comprising one or more branchings at the center of the alkyl group. Preferred ethersulfates are the products of ethoxylating linear chain alcohols having 12 to 18 carbon atoms with 2 to 6 ethylene oxide units and subsequent sulfatation.

Another class of suitable anionic surfactants are soaps, such as for example alkali metal salts of lauric acid, myristic acid, palmitic acid, stearic acid or mixtures thereof and example alkali metal salts of natural fatty acid mixtures, such as for example coconut fatty acid, palm kernel fatty acid or tallow fatty acid.

Suitable non-ionic surfactants are for example alkoxylated compounds, in particular ethoxylated and propoxylated compounds. Preferred are condensation products of alkylphenols or fatty alcohols with 1 to 50 equivalents ethylene oxide, propylene oxide or mixtures thereof and in particular condensation products with 1 to 10 equivalents. Another class of suitable non-ionic surfactants are polyhydroxyfatty acid amides with the amide nitrogen substituted by an organic residue carrying one or more hydroxyl groups which may additionally be alkoxylated. A further class of suitable non-ionic surfactants are alkyl glycosides comprising a linear or branched chain alkyl group with 8 to 22 carbon atoms, in particular 12 to 18 carbon atoms, and a mono- or diglycoside unit which is preferably derived from glucose.

Suitable cationic surfactants are for example monoalkoxylated or dialkoxylated quaternary ammonium compounds comprising one or two hydroxyalkyl groups and an alkyl group with 6 to 18 carbon atoms bonded to nitrogen.

The laundry detergent compositions of the present invention may comprise further components, such as for example builders, alkaline components, bleaching agents, bleach activators, enzymes, chelating agents, graying inhibitors, foam inhibitors, brighteners or colorants.

Suitable as builders are all compounds or compositions that are capable of sequestering calcium or magnesium ions from an aqueous solution. Preferred builders are alkali metal phosphates and alkali metal polyphosphates, in particular pentasodium triphosphate; water soluble or water insoluble sodium silicates, in particular layered silicates of the formula Na₅Si₂O₅; zeolites of the structure type A, X and P and mixtures thereof; and trisodium citrate. Organic co-builders may be used in addition to builders, such as for example polyacrylic acid, polyaspartic acid and copolymers of acrylic acid with methacrylic acid, acrolein or sulfonated vinyl monomers and alkali metal salts thereof as well as mixtures thereof.

Suitable alkaline components for laundry detergent compositions of the present invention provide a pH value in the range of 8 to 12 in the aqueous wash liquid at the use concentration of the laundry detergent. Preferred alkaline components are sodium carbonate, sodium sesquicarbonate and sodium metasilicate. Suitable are also other soluble alkali metal silicates.

Suitable bleaching agents for laundry detergent compositions of the present invention are peroxygen compounds, such as alkali metal perborates, alkali metal carbonate perhydrates, alkali metal persilicates, alkali metal persulfates, alkali metal peroxophosphates, alkali metal peroxopyrophosphates, diacyl peroxides, aromatic peroxy acids and aliphatic peroxy acids. Preferred bleaching agents are sodium perborate tetrahydrate, sodium perborate monohydrate, sodium carbonate perhydrate, peroxylauric acid, peroxystearic acid, epsilon-phthalimidoperoxycarboxylic acids, 1,12-diperoxydodecanedioic acid, 1,9-diperoxyazelaic acid and 2-decyldiperoxybutane-1,4-dioic acid. Most preferred are sodium perborate tetrahydrate, sodium perborate monohydrate and coated sodium carbonate perhydrate. Coated sodium carbonate perhydrate suitable for use in liquid detergent compositions is known from WO 2004/056955, which is hereby incorporated by reference.

Suitable bleaching activators for laundry detergent compositions of the present invention are compounds with acyl groups bonded to nitrogen or oxygen atoms, which can undergo a perhydrolysis reaction with hydrogen peroxide in aqueous solution to give a peroxycarboxylic acid. Preferred compounds of this type are peracylated alkylenediamines, in particular tetraacetylethylenediamine (TAED); acylated triazinones, in particular 1,5-diacetyl-2,4-dioxohexahydro-1,3,5-triazine (DADHT); acylated glycolurils, in particular tetraacetylglycoluril (TAGU); N-acylimides, in particular N-nonanoylsuccinimide (NOSI); acylated phenolsulfonates, in particular n-nonanoyloxybenzenesulfonate and iso-nonanoyloxybenzenesulfonate salts (n-NOBS and iso-NOBS); carboxylic acid anhydrides such as phthalic acid anhydride; acylated polyhydric alcohols, such as ethyleneglycol diacetate, 2,5-diacetoxy-2,5-dihydrofuran, acetylated sorbitol and mannitol and acylated sugars, such as pentaacetylglucose; N-acylated lactams, in particular N-acetylcaprolactam, N-acetylvalerolactam, N-nonanoylcaprolactam and N-nonanoylvalerolactam.

A further class of suitable bleaching activators are the nitriles comprising amine or quaternary ammonium groups known from Tenside Surf. Det. 1997, 34(6), pages 404-409, which are hereby incorporated by reference.

Another class of suitable bleaching activators are transition metal complexes capable of activating hydrogen peroxide for stain bleaching. Suitable transition metal complexes are known from EP-A 0 544 490 page 2, line 4 to page 3, line 57; WO 00/52124 page 5, line 9 to page 8, line 7 and page 8, line 19 to page 11, line 14; WO 04/039932, page 2, line 25 to page 10, line 21; WO 00/12808 page 6, line 29 to page 33, line 29; WO 00/60043 page 6, line 9 to page 17, line 22; WO 00/27975, page 2, line 1 to 18 and page 3, line 7 to page 4, line 6; WO 01/05925, page 1, line 28 to page 3, line 14; WO 99/64156, page 2, line 25 to page 9, line 18; and GB-A 2 309 976, page 3, line 1 to page 8, line 32, which are hereby incorporated by reference.

The laundry detergent compositions of the present invention may further comprise enzymes that enhance the cleaning action, preferably lipases, cutinases, amylases, neutral and alkaline proteases, esterases, cellulases, pectinases, lactases and peroxidases and mixtures thereof. The enzymes may be coated or may be adsorbed to one or more carrier components to protect them against loss of enzyme activity.

The laundry detergent compositions of the present invention may also comprise chelating agents which are capable of sequestering transition metal ions and can inhibit the decomposition of peroxygen compounds in the detergent compositions and in the wash liquid during use of the detergent composition. Preferred chelating agents are phosphonic acids, in particular hydroxyethane-1,1-disphosphonate, nitrilotrimethylenephosphonate, diethylenetriamine-penta(methylenephosphonate), ethlyenediamine-tetra(methylenephosphonate) and hexamethylenediamine-tetra(methylenephosphonate); nitrilotriacetic acid; polyaminocarboxylic acids, in particular ethylenediaminetetraacetic acid, diethylenetriaminepentaacetic acid, ethylenediamine-N,N′-disuccinic acid, methylglycindiacetic acid and polyaspartic acid; polyvalent carboxylic acids and hydroxycarboxylic acids, in particular tartaric acid and citric acid; and the alkali metal and ammonium salts of said preferred chelating agents.

The laundry detergent compositions of the present invention may further comprise graying inhibitors which keep soil particles suspended in the wash liquid and inhibit the redeposition of soil onto fibers. Suitable graying inhibitors are for example cellulose ethers, preferably carboxymethylcellulose and alkali metal salts thereof, methylcellulose, hydroxyethylcellulose, hydroxypropylcellulose and polyvinylpyrrolidone.

The laundry detergent compositions of the present invention may also comprise foam inhibitors which reduce foam formation from the wash liquid during use. Suitable foam inhibitors are for example organopolysiloxanes, preferably polydimethylsiloxane, paraffins, waxes, as well as mixtures thereof with small particle silicas. Such foam inhibitors are well known from the prior art.

The laundry detergent compositions of the present invention may also comprise brighteners which can compensate the yellowing of fibers by adsorbing to the fiber, absorbing UV light and reemitting blue light by fluorescence. Suitable brighteners are for example derivatives of diaminostilbenedisulfonic acid, such as 4,4′-bis-(2-anilino-4-morpholino-1,3,5-triazinyl-6-amino)-stilbene-2,2′-disulfonic acid and alkali metal salts thereof or substituted diphenylstyryls, such as 4,4′-bis-(2-sulfostyrlyl)-diphenyl and alkali metal salts thereof.

The laundry detergent compositions of the present invention may further comprise colorants to provide the compositions with a more pleasing appearance.

Laundry detergent compositions of the present invention in the form of liquids or gels may further comprise up to 30 wt.-% of an organic solvent, preferably methanol, ethanol, n-propanol, iso-propanol, n-butanol, ethylene glycol, 1,2-propylene glycol, 1,3-propylene glycol, 1,4-butylene glycol, glycerin, diethylene glycol, ethylene glycol methyl ether, ethanolamine, diethanolamine or triethanolamine or mixtures thereof.

The laundry detergent compositions of the invention impart a stronger and longer lasting fragrance to fabrics treated with said compositions compared to fabrics treated with compositions comprising perfume delivery systems of the prior art. Such laundry detergent compositions may therefore be formulated with a lower amount of fragrant compounds as used in the prior art.

G. Further Uses

The perfume delivery system of the invention may also be used to deliver perfume to other surfaces than fabrics, such as skin, hair or solid surfaces. They may therefore also be used advantageously in personal care products, such as hair shampoos, hair conditioners, body washes, shower gels, soaps, skin care creams and lotions, skin conditioners, sunscreens, deodorants, antiperspirants or color cosmetics. They may further be used in toilet bowl cleaners, toilet bowl gels, car shampoos and rinse aids.

### EXAMPLES

The following examples are provided to illustrate the present invention without intending to limit the scope of the invention.

### Example 1

### Grafting of Silica with an Aminosilane

2 g of 3-aminopropyltriethoxysilane (Dynasilane AMEO) were dissolved in 10 ml deionized water at room temperature. The resulting solution was added in small portions to 20 g precipitated silica Sipemat 22 made by Degussa AG at room temperature while constantly mixing the silica. The resulting product was heated for 12 h at 110° C. in a forced air oven to remove water and complete the grafting reaction.

### Example 2

### Addition of Cationic Polymer to Grafted Silica

5 g of the dry product obtained in example 1 was dry mixed with 0.5 g polyquaternium-10 (Celquat SC-240C made by National Starch & Chemical) and the mixture was heated to 50° C. for 2 h.

### Example 3

### Preparation of Perfume Delivery System

1 g of the dry product obtained in example 2 was placed in a mixer and 2 g of liquid fragrance composition 5862-HBH-LFS made by International Flavors & Fragrances Inc. was added slowly while mixing the grafted silica. A perfume carrier system comprising 67 wt.-% fragrance on carrier was obtained as a free flowing powder.

### Example 4

### Comparative Example

### Addition of Cationic Polymer to Silica

Example 2 was repeated, but untreated precipitated silica Sipernat 22 was used in place of the dry product obtained in example 1.

### Example 5

### Comparative Example

### Preparation of Perfume Delivery System

Example 3 was repeated, but the dry product obtained in example 4 was used in place of the dry product obtained in example 2.

### Example 6

### Fabric Softening Composition

Varisoft WE 16, which is a 90 wt.-% solution in isopropanol of the reaction product of hydrogenated tallow fatty acid with triethanolamine, quaternized with dimethyl sulfate, was used as fabric softening active composition. Varisoft WE 16 comprises N,N-di(tallowyloxyethyl)-N-methyl-N-(2-hydroxyethyl)-ammonium methylsulfate as the major component. 33.2 g Varisoft WE 16 were heated to 40° C. and slowly added to 165.4 g deionized water with stirring. 0.66 g of a 25 wt.-% solution of calcium chloride were added in parallel to control the viscosity of the mixture. The dispersion obtained was cooled to room temperature and 2.09 g of the perfume delivery system prepared in example 3 was added with stirring. The mixture was stirred slowly for a further 2 h to evenly distribute the perfume delivery system in the dispersion. The resulting dispersion contained about 15 wt.-% fabric softening actives and about 0.7 wt.-% fragrance comprised in the perfume delivery system. The dispersion had a pH of 2.5 and a viscosity of 84 cps measured with a Brookfield viscosimeter at room temperature using spindle no. 2. No settling of particles was visible during storage at room temperature for one month.

### Example 7

### Comparative Example

### Fabric Softening Composition

Example 6 was repeated, but the perfume delivery system prepared in example 5 was used in place of the perfume delivery system prepared in example 3.

### Example 8

### Fabric Softening Composition

### Comparative Example

Example 6 was repeated, but 1.44 g of liquid fragrance composition 5862-HBH-LFS made by International Flavors & Fragrances Inc. was used in place of the perfume delivery system prepared in example 3.

Application Testing of Fabric Softening Compositions

Four cotton towels and three sheets of a 50% cotton-50% polyester fabric with a total weight of about 1700 g were washed in a standard Kenmore laundry washer with a cold wash and cold rinse setting using 50 g of 1993 AATCC (American Association of Textile Chemists and Colorists) standard reference detergent. At the beginning of the rinse cycle, 13.5 g of fabric softening composition per kg total weight of fabric was added to the washer. The washed fabric bundle was dried for 1 h in a standard Kenmore laundry drier. The dried cotton towels were stored at room temperature and assessed for fragrance intensity after 12 h and after 7 days storage. Fragrance intensity was evaluated with the pair wise ranking method described in Sensory Evaluation Techniques, M. Meilgaard, G. V. Civille, B. T. Carr, CRC Press, pages 88 to 91, 254 and 268, using a panel of 3 panelists.

The following set of fabric softening compositions was evaluated:

Example 7 (comparative example with untreated silica as carrier)

Example 6 (example according to the invention with silica grafted with organosilane as carrier)

Example 8 (comparative example without carrier)

Results of pair wise rankings are shown in tables 1 and 2 with the numbers indicating the number of rankings where the composition specified in the column label had a stronger fragrance than the composition specified in the row label.

TABLE 1
Fragrance intensity after 12 h

| | Example 7* | Example 6 | Example 8* |
|---|---|---|---|
| Example 7* | | 6 | 0 |
| Example 6 | 0 | | 0 |
| Example 8* | 6 | 6 | |

*Comparative examples

TABLE 2
Fragrance intensity after 7 days

| | Example 7* | Example 6 | Example 8* |
|---|---|---|---|
| Example 7* | | 5 | 0 |
| Example 6 | 1 | | 0 |
| Example 8* | 6 | 6 | |

*Comparative examples

In the rankings, the fabric softening composition of example 6 comprising a perfume delivery system according to the invention provided a level of fragrance significantly higher than the fabric softening composition of example 8 comprising no carrier system or that of example 7 comprising a carrier system of untreated silica. This proves that the perfume delivery system of the invention imparted the fabrics with a more intensive and longer lasting fragrance.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A perfume delivery system comprising
a) water insoluble carrier particles having surface silanol groups, wherein at least part of said silanol groups are substituted with organic residues by grafting with at least one organosilane and wherein at least part of said organic residues carry amino groups,
b) at least one polymer which carries positively charged functional groups and
c) a fragrance adsorbed to or absorbed into said carrier particles.

2. The perfume delivery system of claim 1, wherein the carrier particles comprise silica, a silicate, an aluminosilicate or a mixture thereof.

3. The perfume delivery system of claim 2, wherein the silica is selected from the group comprising of precipitated silicas, fumed silicas and silica gels.

4. The perfume delivery system of claim 2, wherein the aluminosilicate is a zeolite.

5. The perfume delivery system of claim 4, wherein the zeolite is selected from the group comprising of zeolite X, zeolite Y and dealuminated zeolite Y.

6. The perfume delivery system of claim 1, wherein essentially all of the organic residues carry at least one amino group.

7. The perfume delivery system of claim 1, wherein at least part of the amino groups are primary amino groups.

8. The perfume delivery system of claim 1, wherein the positively charged functional groups of said polymer are quaternary ammonium groups.

9. The perfume delivery system of claim 1, wherein the polymer comprises of carbohydrate monomer units.

10. The perfume delivery system of claim 9, wherein the polymer is a modified starch or a modified cellulose.

11. The perfume delivery system of claim 1, wherein the weight ratio of fragrance to carrier particles is from 0.01 to 5.

12. A process for preparing a perfume delivery system according to claim 1, comprising the steps
a) reacting water insoluble carrier particles having surface silanol groups with an organosilane having at least one organic residue comprising at least one amino group to obtain carrier particles, wherein at least part of said silanol groups are substituted with organic residues carrying said amino groups,
b) adding at least one polymer carrying positively charged functional groups to said carrier particles, and
c) contacting the particles obtained in step b) with a fragrance to adsorb said fragrance onto said particles or absorb said fragrance into said particles.

13. The process of claim 12, wherein the organosilane has the formula
(R¹O)₃₋ₙR²ₙSi(CH₂)₃Z
wherein
R¹ and R² are independently methyl, ethyl, n-propyl or n-butyl,
n is 0 or 1,
Z is NR³R⁴,
R³ and R⁴ are independently hydrogen, methyl, ethyl, C₃₋₂₀ alkyl, C₇₋₂₆ aralkyl, (CH₂CH₂O)ₘR⁵ or (CH₂CH₂NH)ₘR⁵,
m is from 1 to 4, and
R⁵ is hydrogen, methyl, ethyl, C₃₋₂₀ alkyl or C₇₋₂₆ aralkyl.

14. A fabric softening composition, comprising a perfume delivery system according to claim 1 and one or more fabric softening active quaternary ammonium compounds.

15. The fabric softening composition of claim 14, comprising one or more fabric softening active quaternary ammonium compounds selected from the group of compounds of formula (I)
R⁶₄₋ₘN⁺[(CH₂)ₙ-Q-R⁷]ₘX⁻ (I)
wherein each R⁶ is independently C₁-C₆ alkyl, C₁-C₆ hydroxyalkyl or benzyl;
R⁷ is independently hydrogen, C₁₁-C₂₂ linear alkyl, C₁₁-C₂₂ branched alkyl, C₁₁-C₂₂ linear alkenyl or C₁₁-C₂₂ branched alkenyl, with the proviso that at least one of R⁷ is not hydrogen;
Q is independently selected from the units having the formula —O—C(O)—, —C(O)O—, —NR⁸—C(O)—, —C(O)—NR⁸—, —O—C(O)—O—, —CHR⁹—O—C(O)— or —CH(OCOR⁷)—CH₂—O—C(O)—, wherein R⁸ is hydrogen, methyl, ethyl, propyl or butyl and R⁹ is hydrogen or methyl;
m is from 1 to 4;
n is from 1 to 4; and
X⁻ is a softener compatible anion.

16. The fabric softening composition of claim 15, wherein in formula (I) R⁶ is methyl; Q is —O—C(O)— or —NH—C(O)—; m is 2 or 3; n is 2; and X⁻ is chloride or methyl sulfate.

17. The fabric softening composition of claim 14, comprising one or more fabric softening active quaternary ammonium compounds selected from the group of compounds of formulae (II) to (VII)
R⁶N⁺[CH₂CHR⁹OH][CH₂CHR⁹OC(O)R⁷]₂ X⁻ (II)
R⁶₂N⁺[CH₂CHR⁹OC(O)R⁷]₂ X⁻ (III)
R⁶N⁺[CH₂CHR⁹OH][CH₂CH₂NHC(O)R⁷]₂X⁻ (IV)
R⁶₂R⁷₂N⁺X⁻ (V)
[R⁷—C(O)NHCH₂CH₂]₂N⁺R⁶[CH₂CH₂OH]X⁻ (VI) 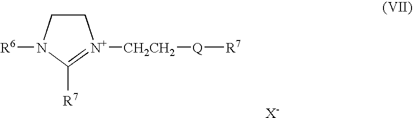
wherein each R⁶ is independently C₁-C₆ alkyl, C₁-C₆ hydroxyalkyl or benzyl;
R⁷ is independently C₁₁-C₂₂ linear alkyl, C₁₁-C₂₂ branched alkyl, C₁₁-C₂₂ linear alkenyl or C₁₁-C₂₂ branched alkenyl;
Q is —O—C(O)— or —NH—C(O)—; and
X⁻ is a softener compatible anion.

18. The fabric softening composition of claim 14, comprising from 0.1 to 5 wt-% of the perfume delivery system, from 1 to 50 wt-% of fabric softening active quaternary ammonium compounds and further comprising water.

19. The fabric softening composition of claim 14, comprising a mixture containing from 0.1 to 5 wt.-% of the perfume delivery system and from 1 to 99 wt.-% of fabric softening active quaternary ammonium compounds disposed on an absorbent article.

20. A laundry detergent composition, comprising a perfume delivery system according to claim 1 and one or more surfactants.
